(19) United States
(12) Patent Application Publication
(10) Pub. No.: US 2019/0314812 A1
(43) Pub. Date: Oct. 17, 2019

(54) SKIN SAMPLE CULTURE AND MEMBRANE TEST DEVICE

(71) Applicant: The University of Dundee, Dundee (GB)

(72) Inventors: Robyn Patricia Hickerson, Balmullo (GB); Michael John Conneely, Dundee (GB)

(21) Appl. No.: 16/302,793

(22) PCT Filed: May 22, 2017
(86) PCT No.: PCT/GB2017/000079

§ 371 Date: Nov. 19, 2018

(30) Foreign Application Priority Data
May 20, 2016 (GB) 1608906.2

(51) Int. Cl. B01L 3/00; C12M 1/42; C12M 1/32; C12M 1/00; C12M 3/00; C12M 1/12; C12N 5/071

(52) U.S. Cl. CPC B01L 3/5085; B01L 2300/0861; C12M 35/04; C12M 23/12; C12M 23/38; C12M 23/46; C12M 23/48; C12M 25/06; C12N 5/0698; B01L 2300/0832; B01L 2300/08; B01L 2300/04; B01L 2200/026; B01L 2300/0618; B01L 2300/0829; B01L 2400/0472; C12N 2503/00; C12N 2503/06; B01L 3/50255

(57) ABSTRACT

A high throughput screening apparatus having a base with channels for receiving a reagent. The channels are spaced across the surface of the base and have one or more walls which extend through the base from a first base surface to a second base surface. A compression member containing a plurality of openings extending through the compression member are positioned across the surface of the compression member, one or more of the openings being positioned for alignment with a corresponding channel in the base. A grip for removably securing the compression member to the base, when a compressible sheet is positioned across the channel between the base and the compression member and fixed by the grip, parts of the compressible sheet are compressed between the base and the compression member to form a seal between the base and the compression member, forming one or more wells for containing the reagent.

### INTRODUCTION

The present invention relates to an apparatus for skin sample culture which is suitable for testing using natural membranes such as skin, synthetic membranes and other materials in sheet form. The present invention relates in particular, to a multiwell skin cell culture device which is suitable for use in high throughput screening.

### BACKGROUND TO THE INVENTION

Mammalian skin is composed of two primary layers, the epidermis and the dermis. In order for skin to retain its normal appearance and to function fully in a normal manner, both layers of the skin need to be present.

The epidermis is composed of the outermost layers of the skin. It forms a protective barrier over the body's surface, is responsible for keeping water in the body, protecting from UV light and preventing pathogens from entering. The epidermis contains no blood vessels and cells in the deepest layers are nourished by diffusion from blood capillaries extending to the upper layers of the dermis.

The dermis is the layer of skin beneath the epidermis; it comprises connective tissue and cushions the body from stress and strain. The dermis provides tensile strength and elasticity to the skin through an extracellular matrix composed of collagen fibrils, microfibrils, and elastic fibers. The dermis is tightly connected to the epidermis through a basement membrane and is structurally divided into two areas: a superficial area adjacent to the epidermis, called the papillary region, and a deep thicker area known as the reticular region.

Samples of skin may be removed from an animal body for the purpose of analysis or in order to grow a sample of skin where a skin graft is required.

Analysis may be undertaken using high throughput screening (HTS)—a technique which is used extensively in drug discovery, biology and chemistry. A typical HTS is performed in a multi-well plate containing target molecules and/or cells. Using robotics, data processing and control software, liquid handling devices, and sensitive detectors, HTS allows a researcher to quickly conduct millions of chemical, genetic, or pharmacological tests. Through this process one can rapidly identify active compounds, antibodies, or genes that modulate a particular biomolecular pathway. The results of these experiments provide starting points for drug design and for understanding the interaction or role of a particular biochemical process in biology.

A multiwell plate is typically a flat plate with multiple wells which function as small test tubes. The multiwell plates used for HTS typically have 96, 384 or 1536 sample wells arranged in a 2:3 rectangular matrix. Each well typically holds somewhere between tens of nanolitres up to 100 microliters of liquid.

The use of multiwell plates for the analysis of membrane samples is described in WO 2005012549 which discloses an apparatus and method for HTS in which a lamina such as skin is positioned between a donor plate and a receptor plate which has a plurality of wells. The receptor plate and donor plate are both in fluid contact with the lamina and a means for applying an electric current to test the response of the lamina in the presence of test formulations is also provided.

U.S. Pat. No. 6,043,027 describes a multiwell single membrane permeation device which has a top member with apertures, a base member which has a plurality of wells, a membrane sheet upon which cell sample is grown and a gasket which provides a seal between the top member and the membrane sheet.

### SUMMARY OF THE INVENTION

It is an object of the present invention to provide a test device for use with a sample of natural or synthetic sheets and membranes which is suitable for HTS.

It is an object of the present invention to provide a multiwell plate device that allows HTS on tissue, specifically murine or porcine skin tissue and human skin tissue.

It is another object of the present invention to provide a skin culture apparatus which retains a skin sample and maintains the viability of the skin sample in a suitable condition for growth and/or testing.

In accordance with a first aspect of the invention there is provided an apparatus for high throughput screening, the apparatus comprising:

a base comprising a plurality of channels for receiving a reagent, the channels being spaced across the surface of the base and having one or more walls which extend through the base from a first base surface to a second base surface,

a compression member containing a plurality of openings which extend through the compression member and which are positioned across the surface of the compression member, one or more of said openings being positioned for alignment with a corresponding channel in the base;

a grip for removably securing the compression member to the base such that when a compressible sheet is positioned across the channel between the base and the compression member and fixed by the grip, parts of the compressible sheet are compressed between the base and the compression member to form a seal between the base and the compression member and the compressible sheet and walls form one or more well for containing the reagent.

Preferably a rim of the channel on the first base surface or the second base surface is in contact with the rim of the opening.

In use, the apparatus is constructed, then arranged such that the compressible sheet is between the compression member and the base with the compressible sheet forming the bottom surface of a well. A reagent is added to the channels and the reagent, under the action of gravity, is in contact with the compressible sheet at the part which extends across the channel.

Preferably, the rim of the channel which is not in contact with the compressible sheet is open.

Preferably, the compressible sheet is a membrane.

Preferably, the compressible sheet is a natural membrane.

Preferably, the compressible sheet is skin.

Preferably, the skin is murine or porcine skin.

Preferably, the skin is human skin.

Preferably, the compressible sheet is a synthetic membrane.

Preferably, the channel is substantially cylindrical in shape.

Optionally, the channel is substantially cuboid in shape.

Other channel shapes may be used such as conic, or may have a polygonal cross section.

Preferably, the grip comprises one or more fixings which connect the compression plate to the base.

Optionally, the grip comprises a snap fit connection which connects the compression plate to the base.

Optionally, the grip comprises a magnetic connection which connects the compression plate to the base.

Preferably, the base comprises one or more base holes positioned for alignment with one or more corresponding compression plate through holes.

Preferably, the grip comprises a fixing which is sized to connect the one or more compression plate through hole to an aligned base hole.

Optionally, the channel with a skin sample receiving surface upon which at least part skin sample may be placed and which extends across an area defined by the shape of the frame; and

a securing member which is releasably connectable to the base frame and a grip which holds the skin sample under tension.

Preferably, the grip comprises a releasable connection between the base frame and the securing member.

More preferably, the grip comprises one or more fixings which connect the base frame to the securing member.

Optionally, the grip comprises a snap fit connection between the base frame and the securing member.

Optionally, the grip comprises a magnetic connection between the base frame and the securing member.

Preferably, the base comprises one or more base holes positioned for alignment with one or more corresponding compression member through holes.

Preferably, the grip comprises a fixing which is sized to connect the one or more compression member through holes to aligned base holes.

Preferably, the grip provides a substantially even tensile force across the skin sample.

Preferably, the apparatus further comprises a tensioner which applies a tensile force across the surface of the compressible sheet.

Preferably the tensioner applies a substantially constant tension across the surface of the compressible sheet.

Optionally, spacers inserted between the compressible sheet and the compression member can be used to optimise the tension across the surface of the compressible sheet.

Optionally, a pattern designed on the compression sheet can be used to optimise the tension across the surface of the compressible sheet.

Preferably, the apparatus further comprises one or more spacer which sets the distance between the base and the compression member.

Optionally, the one or more spacer creates a distance between the base and the compression member which is substantially uniform across the surface of the apparatus.

Optionally, the one or more spacer creates a distance between the base and the compression member which is greater at one part of the apparatus than at another.

Preferably, the one or more spacer is positioned between the base and the compression member.

Preferably, the spacer is in contact with the compression member and the compressible sheet

Preferably, the apparatus further comprises a fluid cap for introducing a fluid into the well.

Preferably, the fluid cap is positioned on the base at the end of the well remote from the compression member.

Preferably, the fluid cap comprises an inlet located at a first position on the fluid cap and an outlet located at a second position on the fluid cap.

Preferably, the fluid cap is adapted to receive a gas.

Optionally, the fluid cap is adapted to receive a liquid.

In accordance with a second aspect of the present invention there is provided a method for conducting high throughput screening using the apparatus in accordance with the first aspect of the invention, the method comprising the steps of:

Placing a compressible sheet between a base and a compression member;

Securing the compressible sheet in position;

Inverting the apparatus;

Adding reagent to the well formed by the channel and compressible sheet.

In accordance with a third aspect of the invention there is provided a method for conducting high throughput screening using the apparatus in accordance with the first aspect of the invention, the method comprising the steps of:

Placing a compressible sheet between a base and a compression member; Securing the compressible sheet in position;

reagent to either surface of the compressible sheet before inverting the apparatus and adding culture medium to the well.

Optionally, the apparatus is constructed to ANSI/SBS dimension standards offers advantages for compatibility with currently available automated handling apparatus.

### BRIEF DESCRIPTION OF THE DRAWINGS

The present invention will now be described with reference to the accompanying drawings in which:

FIG. 1 shows an exploded perspective view of a first embodiment of a skin sample culture and membrane test device in accordance with the present invention;

FIG. 2 a plan view of the embodiment of FIG. 1;

FIGS. 3A, 3B and 3C are cross sectional view of the embodiment of FIG. 1 and illustrate the process of using the apparatus as a multi-well plate; and

FIGS. 4A and 4B are examples of suitable compressible materials.

FIG. 5A is a perspective view of a second embodiment of the present invention and FIG. 5B is a side view of the embodiment in FIG. 5A;

FIG. 6 shows a perspective view of another embodiment of a skin sample culture and membrane test device with a fluid cap in accordance with the present invention;

FIG. 7 is a plan view of an apparatus in accordance with the present invention, in which leakage from well to well has been measured;

FIG. 8A is a perspective view of another embodiment of the present invention, FIG. 8B illustrates experimental data in which skin cultured in the present invention responds to a small molecule drug and FIG. 8C is a graph which plots NQO1 mRNA levels versus time of treatment; and

FIG. 9 shows an embodiment of the present invention which is designed to standard ANSI dimensions.

### DETAILED DESCRIPTION OF THE DRAWINGS

High throughput screening (HTS) is the core of drug discovery. A typical HTS is performed in 384-well plates containing target molecules and/or cells. Here we describe a plate device that allows HTS on tissue, specifically murine or porcine skin tissue and human skin tissue obtained from abdominoplasty surgery.

The present invention comprises, a base and matching compression plate between which the compressible sheet is placed.

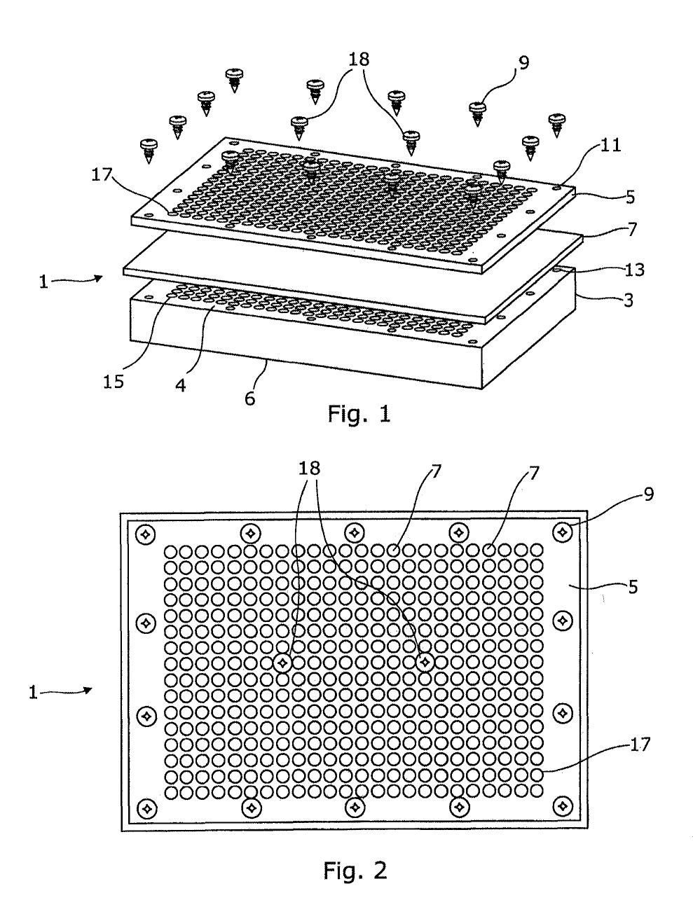

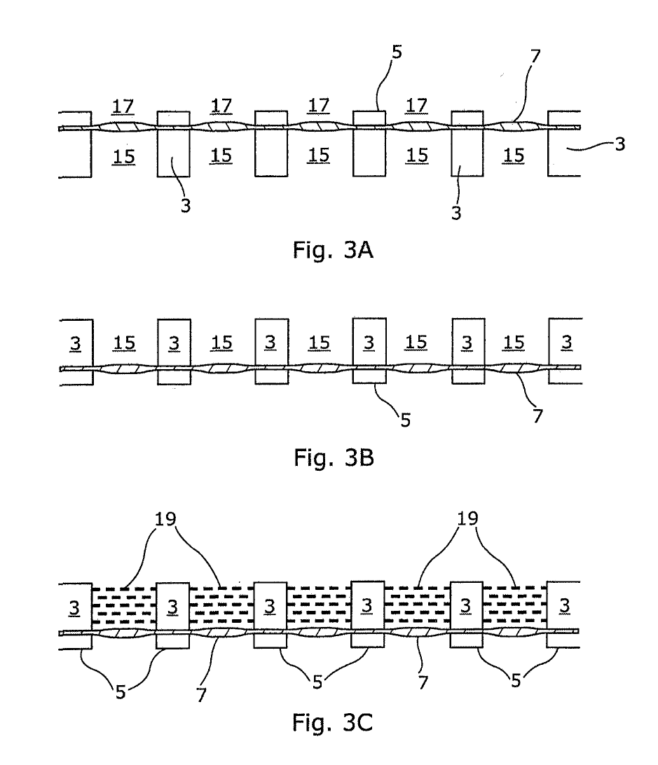

FIG. 1 is an exploded perspective view of a first embodiment of the present invention. FIG. 2 is a plan view of the same and FIG. 3 is a cross section of part of the device. An embodiment of the present invention will be described with reference to FIGS. 1 to 3.

FIGS. 1 to 3 show an apparatus 1 which comprises a base 3, a compression plate 5 and a compressible sheet 7 which is positioned between the base 3 and the compression plate 5. The base comprises an array of channels 15 which are substantially cylindrical in cross section and extend from a first surface 4 which, in this example is positioned towards the compression plate 5, to a second surface 6 which is remote from the compression plate 5. The channel is open ended at the first surface 4 and at the second surface 6 which defines a circular rim or lip.

The compression plate 5 comprises a substantially planar member which has a plurality of openings 17 arranged in an array. The size and position of the openings 17 matches the size and position of the channels 15 in the base 3 such that when the compression plate 5 is aligned with and placed upon the base 3, the openings 17 of the compression plate 5 and the channels 15 of the base 3 are aligned to have a common centre point.

A grip mechanism is included in order to secure the compression plate 5 to the base 3. In this example, the grip comprises a series of screws 9 which are connectable to the compression plate via through holes 11 and is connectable to the base via base holes 13.

In this example, screws 9 fasten together the compression plate 5, compressible sheet 7 and base 3 around the perimeter of the compression plate 5 and base 3. Additional screws 18 are used towards the centre of the compression plate as shown in FIG. 2.

When the compression plate 5 is secured through the compressible sheet 7 to the base 3, the parts of the compressible sheet 7 between the base and the compression member are compressed to form a seal between the base and the compression member and the compressible sheet and walls forms a well for containing the reagent. As is shown in FIG. 3A. In this example of the present invention, the compressible material is porcine or human skin, that single sample acting as a gasket between the upper and lower plates.

In use, a single skin sample 7, is placed across all wells 15 of base 3 either unstretched or under a user defined tension. Once the skin 7 is in place and the compression plate 5 is secured with the screws 9, the skin 7 acts as a gasket. As shown in FIGS. 3B and 3C, the entire device can then be turned over and the wells 15 filled with a reagent medium 19 (with or without test compound). The entire device is then incubated in the “upside down” orientation in a standard incubator with appropriate secondary containment. Breathable plate seals can be used with this plate.

Optionally, prior to turning over the device and filling wells 15 with reagent medium a topical treatment to the membrane 7 can be applied.

The clamping force compresses the skin 7 in between each channel 15, effectively sealing each channel 15 using the skin itself to form the well. In this manner each well can be considered a discrete sample where an individual experiment can be performed.

In some embodiments of the present invention the skin is tensioned before clamping the top plate in place. It has been noted that the act of compressing the skin around each channel without additional tensioning causes the free skin over the well opening to bulge into the opening. This seems to stretch the piece of free skin to a degree sufficient enough to maintain it in culture. Tensioning is preferred where the channel cross sectional area is large.

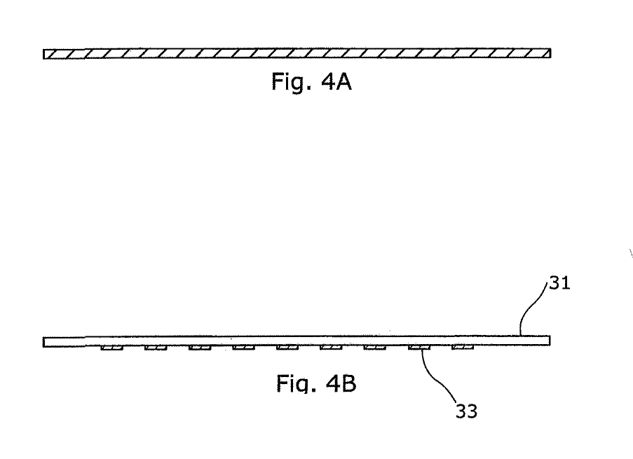

FIGS. 4A and 4B show examples of a compressible sheet. FIG. 4A shows a membrane such as porcine or murine skin. FIG. 4B shows a compressible substrate 31 upon which a sample 33 may be mounted.

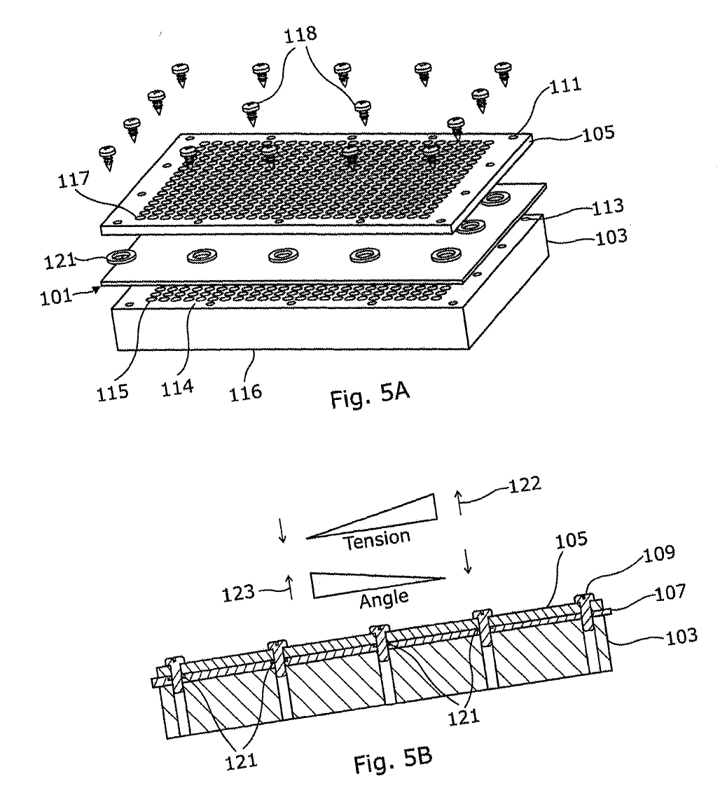

FIG. 5A shows a perspective view of another embodiment of the present invention with the addition of optional spacers 121. In use the spacers 121 are inserted between the compression member 5 and the compressible sheet or sample of skin 7.

FIGS. 5A and 5B show an apparatus 101 which comprises a base 103, a compression plate 105 and a compressible sheet 107 which is positioned between the base 3 and the compression plate 5. The base comprises an array of channels 115 which are substantially cylindrical in cross section and extend from a first surface 114 which, in this example is positioned towards the compression plate 105, to a second surface 116 which is remote from the compression plate 105. The channel is open ended at the first surface 114 and at the second surface 116 which defines a circular rim or lip.

The compression plate 105 comprises a substantially planar member which has a plurality of openings 117 arranged in an array. The size and position of the openings 117 matches the size and position of the channels 115 in the base 103 such that when the compression plate 5 is aligned with and placed upon the base 103, the openings 117 of the compression plate 105 and the channels 115 of the base 103 are aligned to have a common centre point.

FIG. 5B shows spacers 121 of varying thickness which have been compressed between the compressible member 105 and the compressible sheet 107. The spacers 121 bear some of the compression load experienced by the compressible sheet 107 and the base 103 when the compression plate 105 is tightened into position. Therefore, the spacers 121 function as a means of controlling the compression on the compressible sheet 107 and the tension across the surface of the sheet where it forms part of a well.

In this example, a gradient of tension 122 and compression has been applied to the skin sample through the use of the spacers which imparts a slight angle 123 to the compressible member 105.

In another embodiment, a varied number of spacers of differing thicknesses can be used to optimize or change the compression and tension.

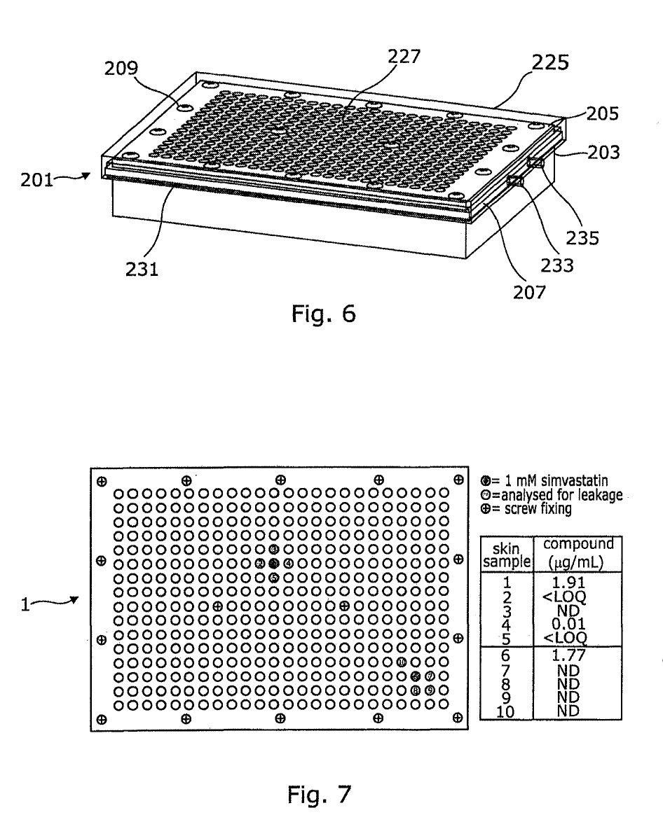

FIG. 6 is a perspective view of another embodiment of the present invention. FIG. 6 shows the apparatus 201 with a base 203, a compression plate 205 and a fluid cap 225. The base has holes (not shown) which receive fixings 209. The base is rectangular in shape and further comprises channels (not shown) which extend through the perimeter of the base, allowing the culture medium, air and other fluids to move through the space at or below the underside of the skin sample 207.

The fluid cap 225 is substantially rectangular in shape having an enclosed top surface 227, an enclosed side surface 229 with a seal 231 on its lower perimeter. The seal is designed to retain the fluid in the space at or around the top surface of the skin sample 207. The inlet 233 is connectable to a fluid source and the outlet 235 is connected to a fluid collector. In use, the fluid cap 225 is placed over the compression plate 205 and pushed downwards into place and the seal 231 holds the fluid cap in position. A fluid source is connected to the fluid cap inlet 233. The fluid may be introduced as a batch into the fluid cap 225, in which the outlet 235 is closed and once the required amount of fluid has been added, the inlet 233 is closed. Alternatively, the fluid may be introduced continuously so a continuous flow of fluid passes through the fluid cap 225, in this case the inlet 233 and the outlet 235 remain open, the outlet 235 being connected to a fluid collection vessel (not shown).

The fluid cap will allow the ability to culture skin such that the atmosphere (e.g., humidity, gas composition, etc.) at the surface of the skin can be controlled separately from the atmosphere of the incubator.

FIG. 7 is a plan view of an apparatus in accordance with the present invention, in which leakage from well to well has been measured. In wells labelled 1 and 6, medium containing 1 mM simvastatin was added at time 0 h. All other wells contained medium only. After 24 h, the skin in wells 1-10 was removed from the device using a 3 mm biopsy punch. The skin was homogenised in organic solvent to extract simvastatin from the treated and peripheral skin. Simvastatin levels in the solvent were then quantitated by LC-MS. High levels of simvastatin were measured in wells 1 and 6 which were treated with simvastatin directly. Little (<0.6%) or no leakage was detected in the neighbouring wells. <LOQ=below the limit of quantitation. ND=not detected.

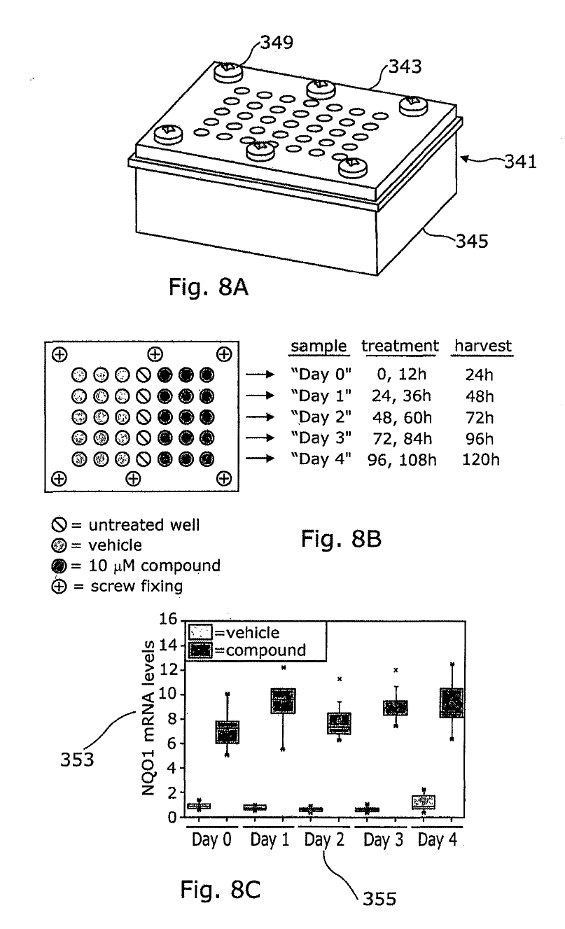

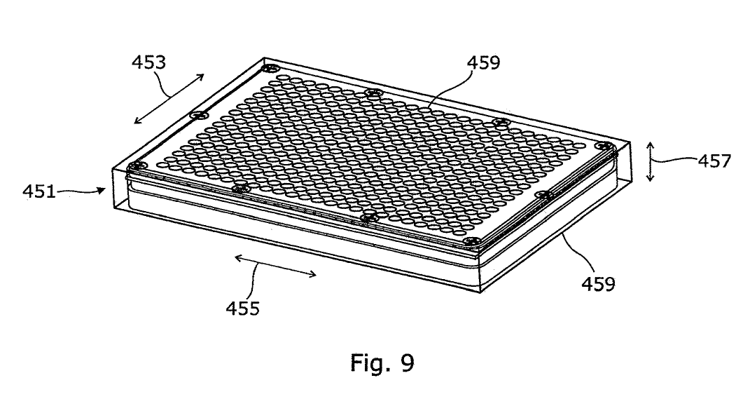

FIG. 8A is a perspective view of another embodiment of the present invention 341. FIG. 8A shows the apparatus 341 with a base 345, and compression plate 343. The base has holes (not shown) which receive fixings 349. FIG. 8A shows a partial 384-well device 341 that has the overall well dimensions and spacing of a standard 384-well plate used for HTS. FIG. 8B is a schematic diagram which summarises an experiment in which skin cultured in the apparatus 341 was treated with a small molecule NRF2 activator (TBE-31) in order to evaluate the intracellular viability of the tissue. NRF2 transcriptionally regulates multiple genes that play both direct and indirect roles in activating intracellular anti-oxidative pathways.

One of the genes that is upregulated upon NRF2 activation is NAD(P)H dehydrogenase [quinone] 1 (NQO1). FIG. 8C shows NQO1 mRNA levels 353 obtained from total RNA isolated from the skin treated with TBE-31 compound (a potent NRF2 activator) at the indicated times 355. It is shown here that the intracellular viability of the skin cultured in this device remains viability for at least 4 days as the NRF2 response is statistically the same whether the tissue is treated on Day 0 or Day 4.

FIG. 9 is a perspective view of another embodiment of the present invention. FIG. 9 shows the apparatus 451 which is designed in accordance with ANSI/SBS/SLAS dimensions. Well 459 spacing and apparatus footprint including, width 453, length 455 and height 457 all match standard microplate dimensional standards to ensure compatibility with automated handling systems.

The device of the present invention can be machined or 3D printed in a variety of materials, including but not limited to plastics such as ABS, Polypropylene, Polystyrene, PTFE, PEEK or PET and metals such as stainless steel or titanium. Additionally, the device can be mass produced through methods such as injection moulding, insert moulding and vacuum forming.

The compression plate can be secured using a variety of methods (depending on application and design), including, but not limited to, screw, spring clip and magnetic fixation. The device holds the membrane under a user-defined tension at the air-liquid interface and allows it to be maintained in culture in a format featuring a footprint and well spacing matching ANSI/SBS standard dimensions, affording the device compatibility with various automated methods of handling. The designed number of wells can range from 12 to 384, preferably arranged in a 2:3 rectangular matrix and the thickness of the plates can range from 2 mm to 15 mm depending on application.

Each well is separate, containing its own volume of culture medium. The medium is added through standard automated pipettes which are a standard part of high throughput screening apparatus. Because the plate is handled upside down and each well is separately filled, this and other embodiments of the invention have no single reservoir of culture medium and thus no requirement to allow air to escape.

In another embodiment of the invention, the base may be submerged in a reagent medium prior to attachment of the compression plate in order to fill each well with the same solution. In most cases, different reagents may be used in each well or triplicate of wells as they will contain different compounds dissolved at different concentrations in medium.

Improvements and modifications may be incorporated herein without deviating from the scope of the invention.

1. An apparatus for high throughput screening, the apparatus comprising:
a base comprising a plurality of channels for receiving a reagent, the channels being spaced across the surface of the base and having one or more walls which extend through the base from a first base surface to a second base surface;
a compression member containing a plurality of openings which extend through the compression member and which are positioned across the surface of the compression member, one or more of said openings being positioned for alignment with a corresponding channel in the base; and
a grip for removably securing the compression member to the base such that when a compressible sheet is positioned across the channel between the base and the compression member and fixed by the grip, parts of the compressible sheet are compressed between the base and the compression member to form a seal between the base and the compression member and the compressible sheet and walls of the base form one or more wells for containing the reagent.

2. (canceled)

3. An apparatus as claimed in claim 1 wherein, the compressible sheet is a natural membrane or a synthetic membrane.

4. An apparatus as claimed in claim 1 wherein, the compressible sheet is skin.

5. An apparatus as claimed in claim 4 wherein, the skin is human, murine or porcine skin.

6. (canceled)

7. An apparatus as claimed in claim 1 wherein, the channel is substantially cylindrical in shape.

8. An apparatus as claimed in claim 1 wherein, the channel is substantially cuboid, conic, or has a polygonal cross section.

9. An apparatus as claimed in claim 1 wherein, the grip comprises one or more fixings which connect the compression plate to the base.

10. An apparatus as claimed in claim 1 wherein, the grip comprises a snap fit connection which connects the compression plate to the base.

11. An apparatus as claimed in claim 1 wherein, the grip comprises a magnetic connection which connects the compression plate to the base.

12. An apparatus as claimed in claim 1 wherein, the base comprises one or more base holes positioned for alignment with one or more corresponding compression plate through holes.

13. An apparatus as claimed in claim 1 wherein, the grip comprises a fixing which is sized to connect the one or more compression plate through hole to an aligned base hole.

14. An apparatus as claimed in claim 1 wherein, the grip provides a substantially even tensile force across the skin sample.

15. An apparatus as claimed in claim 1 wherein, the apparatus further comprises a tensioner which applies a tensile force across the surface of the compressible sheet.

16. An apparatus as claimed in claim 1 wherein, the apparatus further comprises one or more spacer which sets the distance between the base and the compression member.

17. An apparatus as claimed in claim 16 wherein, the one or more spacer creates a distance between the base and the compression member which is substantially uniform across the surface of the apparatus.

18. An apparatus as claimed in claim 16 wherein, the one or more spacer creates a distance between the base and the compression member which is greater at one part of the apparatus than at another.

19. An apparatus as claimed in claim 16 wherein, the one or more spacer is positioned between the base and the compression member.

20. (canceled)

21. An apparatus as claimed in claim 1 wherein, the apparatus further comprises a fluid cap for introducing a fluid into the well.

22. (canceled)

23. An apparatus as claimed in claim 21 wherein, the fluid cap comprises an inlet located at a first position on the fluid cap and an outlet located at a second position on the fluid cap.

24. (canceled)

25. (canceled)

26. A method for conducting high throughput screening using the apparatus in claimed in claim 1 the method comprising:
placing a compressible sheet between a base and a compression member;
securing the compressible sheet in position;
inverting the apparatus; and
adding reagent to the well formed by the channel and compressible sheet.

27. A method for conducting high throughput screening using the apparatus claimed in claim 1, the method comprising:
placing a compressible sheet between a base and a compression member;
securing the compressible sheet in position; and
applying reagent to either surface of the compressible sheet before inverting the apparatus and adding culture medium to the well.

28. The method according to claim 27, further comprising adding a reagent to the culture medium.
